(19) United States
(12) Patent Application Publication
(10) Pub. No.: US 2026/0014347 A1
(43) Pub. Date: Jan. 15, 2026

(54) INTERMITTENT CATHETER

(71) Applicant: ConvaTec Limited, Flintshire (GB)

(72) Inventors: Marian Novak, Trebisov (SK); Oliver Pfleger, Wirral (GB)

(21) Appl. No.: 19/330,297

(22) Filed: Sep. 16, 2025

Related U.S. Application Data
(63) Continuation of application No. PCT/GB2024/050719, filed on Mar. 15, 2024.
(60) Provisional application No. 63/452,854, filed on Mar. 17, 2023.

(30) Foreign Application Priority Data
Apr. 4, 2023 (GB) 2305009.9

(51) Int. Cl. A61M 25/00

(52) U.S. Cl. CPC A61M 25/0017; A61M 25/0009; A61M 25/002; A61M 25/0045

(57) ABSTRACT

A catheter wetting agent storage chamber defined by a first end wall, a second end wall and a radially outer wall extending along a longitudinal axis between the two. The catheter wetting agent storage chamber has a first section with a first part of the outer wall and the first end wall and a second section comprising a second part of the outer wall and the second end wall. One of the first and second section is disposed radially inside the other section such that the sections overlap axially. One of the first and second sections comprises at least one projection which extends axially into a corresponding recess in an outer surface of the outer wall of the other of the first and second sections.

### TECHNICAL FIELD OF THE INVENTION

The present invention relates to an intermittent catheter (e.g. a urinary catheter).

### BACKGROUND TO THE INVENTION

A catheter is a medical device comprising a hollow catheter tube designed for insertion into canals, vessels, passageways or body cavities to permit injection, drainage or withdrawal of fluids or substances therefrom, or to ensure said canals, vessels, passageways etc. remain open. Urinary catheters are designed for use for insertion into a user's bladder via the urethra to drain the bladder.

To maximise comfort and minimise the risk of trauma and/or infection, an outer surface of the catheter tube is typically wetted using a wetting agent prior to insertion by the user. In further developments, the catheter tube itself comprises, is integrated with or is coated with a hydrophilic component (e.g. a hydrophilic polymer) which serves to reduce friction further upon application of the wetting agent.

Some catheters may be supplied pre-wetted in a packaging, for instance, where the catheter is at least partially submerged within wetting agent within the packaging. Whilst this may ensure the catheter tube is adequately wetted prior to use, such arrangements suffer in that components of the catheter other than the catheter tube such as a gripper element or funnel can also become wetted. This has a detrimental effect of the experience of the user where it may become difficult to hold and direct the catheter tube as required. This is particularly problematic where the user is performing self-catheterisation. Further, having the catheter submerged may effectively reduce the shelf-life of the catheter due to long-term exposure of components of the catheter to moisture.

It is therefore seen as advantageous to provide a catheter which may be wetted at or immediately prior to the point of use.

In an attempt to address this, some catheters are provided in packaging which is configured to release wetting agent upon actuation of a mechanism. The mechanisms of the prior art can be actuated in different ways, in some instances rotation of an element of the packaging activates the wetting action whereas in other instances it is an axial motion. Mistakenly using the wrong motion in an attempt to active the wetting action may cause damage to the catheter packaging and frustrate the user.

It is an aim of an embodiment or embodiments of the invention to overcome or at least partially mitigate one or more problems with the prior art and/or to provide an improved intermittent catheter.

### SUMMARY OF THE INVENTION

The present invention provides a catheter assembly according to the appended claims.

An aspect of the present disclosure provides a catheter wetting agent storage chamber defined by a first end wall, a second end wall and a radially outer wall extending between the two. The radially outer wall may extend along a radial axis. The catheter wetting agent storage chamber may comprise a first section comprising a first part of the outer wall and the first end wall. The catheter wetting agent storage chamber may comprise a second section comprising the second end wall and a second part of the outer wall. One of the first and second sections may be disposed radially inside the other section. The first and second sections may overlap axially. One of the first and second sections may comprise at least one projection. The at least one projection may extend axially into a corresponding recess in an outer surface of the outer wall of the other of the first and second sections.

An aspect of the present disclosure provides a catheter wetting agent storage chamber defined by a first end wall, a second end wall and a radially outer wall extending along a longitudinal axis between the two, wherein the catheter wetting agent storage chamber comprises: a first section comprising a first part of the outer wall and the first end wall, a second section comprising the second end wall and a second part of the outer wall; wherein one of the first and second section is disposed radially inside the other section such that the sections overlap axially; and wherein one of the first and second sections comprises at least one projection which extends axially into a corresponding recess in an outer surface of the outer wall of the other of the first and second sections.

Advantageously, where the catheter wetting agent storage chamber is provided in at least two sections, providing corresponding projections and recesses allows for a discontinuous circumferential joint between the two sections. This discontinuous joint is visible in normal use, which provides a visual prompt to the user or health care professional (HCP) that the chamber is not to be rotated.

The second section may be disposed inside the first section. The first section may be disposed inside the second section. The second section may comprise the at least one projection.

The catheter wetting agent storage chamber may comprise an equal number of projections and recesses. The catheter wetting agent storage chamber may comprise two projections and two recesses. The catheter wetting agent storage chamber may comprise three projections and three recesses. The catheter wetting agent storage chamber may comprise four projections and four recesses. The or each recess may extend through the outer wall of the corresponding section. The or each recess may extend radially through the outer wall of the corresponding section. The or each projection may be provided on an outer surface of the outer wall. The or each projection may be provided on an outer surface of the outer wall of the corresponding section.

The or each projection may have the same profile as the or each recess. The or each projection may have the same profile as the or each corresponding recess.

The projection and recess both having the same profile provides for a clean joint line between the two portions, this improves the aesthetics of the chamber.

The second part of the outer wall may have a distal section with a first diameter. The second part of the outer wall may have a proximal section with a second diameter. The first diameter may be less than the second diameter. The first diameter may be equal to an inner diameter of the first wall. The second diameter may be equal to an outer diameter of the first wall. The projections may have the second diameter.

The projections may have curved distal end. The projections may have a textured outer surface. A textured surface allows for improved grip by the user of HCP.

The first and second sections may engage with a snap fit. The first and second sections may engage with a cantilever snap fit. The cantilever snap fit may comprise corresponding projections and recesses. The snap fit may comprise radially extending projections. The snap fit may comprise radially extending recesses. The snap fit may comprise a radially extending projection on one of the first and second sections and the other of the first and second sections may comprise a corresponding recess.

A cantilever snap fit provides a strong engagement between the two components but need not allow for rotational movement between the two portions of the chamber, the cantilevered projections may be susceptible to damage if an attempt to rotate is made, thus provision of corresponding projections and recesses reduces the risk of damage.

The first and second sections may comprise complimentary alignment features. One of the first and second sections may comprise one or more slits and the other of the first and second sections may comprise one or more projections. The one or more slits may comprise a tapered opening. The one or more projections may comprise ribs. The ribs may be provided on the first section. The ribs may be provided on a radially inner surface of the first section. The ribs may be provided on a radially inner surface of the first part of the outer wall. The ribs may extend axially. The slit may be provided on the second section. The slit may extend through a extend through the second part of the outer wall.

The catheter wetting agent storage chamber may be a wetting chamber. The wetting chamber may be provided in an external housing. The catheter wetting agent storage chamber may comprise one or more filling apertures. The one or more filling apertures may be configured to be sealed when the first and second sections are joined.

The wetting agent storage chamber may be further defined by a movable insert. The movable insert may be movable with respect to the radially outer wall between a first position and a second position. In the first position the movable insert may be aligned with a chamber wall to seal the storage chamber. In the second position the movable insert may be misaligned with the chamber wall to open the catheter wetting agent storage chamber. Removal of a cap may move the movable insert from the first position to the second position. The chamber wall may be the first end wall, the second end wall and/or the radially outer wall.

A movable insert provides an alternative way of deploying the catheter without rotation.

The wetting agent storage chamber may comprise a movable insert. The wetting chamber may further comprise a movable insert. The movable insert may comprise one or more ribs extending circumferentially. The ribs may extend to the chamber wall. The movable insert may comprise at least two ribs. The movable inserter may comprise at least one upper rib and at least one lower rib. For example, the inserter tip may comprise a pair of upper ribs and a pair of lower ribs. Alternatively (or additionally) the chamber wall may comprise one or more ribs extending circumferentially. The chamber wall ribs may extend to the inserter tip. The chamber wall may comprise two ribs. One of the inserter tip or chamber wall preferably comprises one or more ribs extending circumferentially and projecting to the other of the chamber wall or insert.

The movable insert may be configured to move between a first position and a second position. The movable insert may be configured to move axially between a first position and a second position. The movable insert may be movable between a first position and a second position. The movable insert may be axially movable between a first position and a second position. The first position may be a sealed position. The second position may be a catheter wetting position. In the first position the catheter wetting agent storage chamber may be sealed. In the second position the catheter wetting agent storage chamber may be open.

In a first position the one or more ribs may define an axial extent of the wetting agent storage chamber. Where the movable insert comprises upper and lower ribs, the distance between the upper and lower ribs may define the axial extent of the storage chamber. Where the chamber wall comprises two ribs, the two ribs may define the axial extent of the storage chamber.

In the first position one or more or each rib of the inserter tip preferably aligns with a corresponding sealing surface on the chamber wall to seal the storage chamber. Alternatively, in the first position one or more or each rib of the chamber wall preferably aligns with a corresponding sealing surface on the movable insert to seal the storage chamber. In the second position at least one rib of the movable insert is preferably axially misaligned with a sealing surface on the chamber wall. Where the assembly comprises upper and lower ribs, the upper rib may remain aligned with a sealing surface on the chamber wall in the second position. In an alternative, in the second position at least one rib of the chamber wall is axially misaligned with a sealing surface on the movable insert.

The storage chamber may be sealed with a seal element. One or more of the ribs may comprise a seal element. The seal element may be integrally formed with the inserter tip. The seal element may be integrally formed with the external housing. The seal element may be mounted between a pair of ribs.

The seal element may be an elastomeric material. For example, the seal may be a rubber seal or some other suitable material. The seal element may be an annular seal element. The seal element may comprise an O-ring. The seal element may comprise an X-ring. The seal element may comprise a U-cup. The seal element may lie in a radial plane, for example, the normal plane of the longitudinal axis of the catheter assembly. The seal element may extend between radially opposed sealing surfaces. The O-ring may lie in a radial plane, for example, the normal plane of the longitudinal axis of the catheter assembly. The seal element may be provided in the form of a gasket.

The seal element may be compressible.

The seal element may be located in a seal element housing in one of the chamber wall or the movable insert. The seal element housing may comprise a groove or channel provided within the chamber wall or movable insert. The groove or channel may be provided in part by a radially outer wall, an axial end wall and/or one or more catheter guide features provided within the storage chamber. The seal element may be overmoulded so as to become an integral part of the storage chamber or inserter tip. One or more of the sealing surfaces may be provided by the seal element housing. The at least one sealing surface may slideably oppose the seal element. The at least one sealing surface may be referred to as a primary sealing surface.

At least one of the movable insert and the chamber wall may comprise a divergent portion over which the rib passes when transitioning between the first position and second position such that the distance between the chamber wall and inserter tip at the axial location of the rib is increased when in the second position when compared to the first position. The distance may be a radial distance with respect to the longitudinal axis. The divergent portion may comprise a widening of a cavity adjacent to the seal surface. The cavity may be that of the storage chamber and/or an adjacent chamber. The adjacent chamber may be provided within the external housing. The adjacent chamber may house a catheter tube. The adjacent chamber may be a priming chamber or a wetting chamber.

The divergent portion may comprise a step, taper or chamfer in the wall surface which is adjacent to the sealing surface. The sealing surface may comprise a cylindrical surface having a first diameter. An adjacent wall portion of the chamber wall or inserter tip may have a second diameter which is different to the first diameter. The first diameter may be greater than the second diameter. The adjacent wall portion may be proximal an insertion end of the catheter. The adjacent wall portion may be a secondary surface.

The catheter wetting chamber may further comprise a wetting agent. In the first position the wetting agent may be sealed within the wetting agent storage chamber. When in the second position, a flow path may be provided between the sealing surface and rib such that the wetting agent may flow out of the storage chamber when in the second position. The flow path may direct the wetting agent out of the storage chamber to a catheter.

The catheter wetting agent storage chamber may be provided in a catheter assembly. The catheter assembly may further comprise a catheter.

Accordingly, another aspect of the present disclosure provides a catheter assembly comprising a catheter and a catheter wetting agent storage chamber defined by a first end wall, a second end wall and a radially outer wall extending between the two. The radially outer wall may extend along a radial axis. The catheter wetting agent storage chamber may comprise a first section comprising a first part of the outer wall and the first end wall. The catheter wetting agent storage chamber may comprise a second section comprising the second end wall and a second part of the outer wall. One of the first and second sections may be disposed radially inside the other section. The first and second sections may overlap axially. One of the first and second sections may comprise at least one projection. The at least one projection may extend axially into a corresponding recess in an outer surface of the outer wall of the other of the first and second sections.

An aspect of the present disclosure provides a catheter assembly comprising a catheter and a catheter wetting agent storage chamber defined by a first end wall, a second end wall and a radially outer wall extending along a longitudinal axis between the two, wherein the catheter wetting agent storage chamber comprises: a first section comprising a first part of the outer wall and the first end wall, a second section comprising the second end wall and a second part of the outer wall; wherein one of the first and second section is disposed radially inside the other section such that the sections overlap axially; and wherein one of the first and second sections comprises at least one projection which extends axially into a corresponding recess in an outer surface of the outer wall of the other of the first and second sections.

Advantageously, where the catheter wetting agent storage chamber is provided in at least two sections, providing corresponding projections and recesses allows for a discontinuous circumferential joint between the two sections. This discontinuous joint is visible in normal use, which provides a visual prompt to the user of health care professional (HCP) that the chamber is not to be rotated.

The catheter assembly may further comprise a wetting chamber. In the second position the wetting chamber may be in fluid communication with the storage chamber. The storage chamber and wetting chamber may be separate chambers.

The catheter assembly may further comprise a sheath. A wetting chamber may be at least partially defined by the sheath. The sheath may define a cavity. The catheter may be arranged in the sheath cavity. In the second position the sheath cavity may be in fluid communication with the wetting agent storage chamber. In this regard the sheath cavity may be a wetting chamber.

The catheter may be at least partially arranged within the sheath. The sheath and catheter may define a flow path. The flow path may extend along the axial length of the catheter. The flow path may be provided between an outer surface of the catheter and an inner surface of the sheath.

The catheter assembly may further comprise a storage pouch. The storage pouch may be configured to receive fluid discharge from the catheter in use. In the second position the storage pouch may be in fluid communication with the wetting agent storage chamber. The storage pouch may be in fluid communication with the wetting agent storage chamber via the sheath flow path.

When in the second position, the seal may be separated from at least one of the sealing surfaces so as to provide a flow path for the wetting agent to pass the rib.

The wetting agent may be provided within, or capable of flowing into, the wetting chamber when in the second position.

The catheter may comprise a catheter tube which provides an insertion end for inserting into a patient and an outlet end comprising one or more external handling features.

The catheter may be an intermittent catheter. The catheter may be a urinary catheter. The catheter may be a female intermittent catheter.

The catheter assembly may further comprise a cap. The cap may seal the wetting agent storage chamber. The cap may provide a sterile seal to the wetting agent storage chamber.

The catheter assembly may be configured to provide mechanical feedback to a user. The mechanical feedback may be indicative of the end of a first step. The mechanical feedback may be indicative of the catheter assembly being in the second position. The mechanical feedback may be between the first and second steps.

The provision of a feedback mechanism allows the user to know that the wetting process has occurred without having to visually inspect the catheter.

The catheter may comprise an insertable portion. Prior to the first step the insertable portion may not be in contact with the wetting agent. In a/the first position the catheter may not be in contact with the wetting agent. In a/the second position the wetting agent may be in contact with the catheter.

It is advantageous that prior to deployment of the catheter, for example during shipping and storage, that the insertable portion of the catheter is not in contact with the wetting agent. The insertable portion of the catheter is often made of materials which interact with the wetting agent, this interaction should only occur just prior to use.

The/a cap may be configured to engage with the movable insert in the first position. The/a cap may be configured to disengage from the movable insert in the second position. The/a cap may be releasably coupled to the movable insert.

Advantageously, by making the cap and movable insert releasably coupled, the wetting operation can be initiated in the same action as removing the cap. Furthermore, as the cap and moveable insert only decouple in the second position, the cap can only be removed once in the second position. Movement to the second position may cause wetting of the catheter and as such the cap may only be removed once the wetting action has occurred, therefore reducing the risk of injury to the user associated with use of an unlubricated catheter.

The cap may comprise a deformable portion. The cap may be formed of a resiliently deformable material. The cap may be formed of linear low-density polyethylene. The cap may be formed of low-density polyethylene. The cap may be formed of polypropylene. The cap may be formed of a thermoplastic elastomer. The cap may be formed of thermoplastic polyurethane. The cap may be formed of Ethylene-vinyl acetate.

The cap may be resiliently deformed in the first position. The cap may relax to an original shape in the second position. The cap may alternatively be deformed in the second position.

The cap may comprise an interior cavity. The interior cavity may be shaped to conform to a portion of a movable insert. The cap may comprise a grab loop. The grab loop may be shaped to aid in removal of the cap.

An open end of the cap may be deformable. The open end of the cap may be formed of a deformable material. The open end of the cap may be provided with one or more features to aid in deformation. For example, the open end of the cap may comprise one or more slots extending axially from the open end. The deformation aids may be two slots. The open end of the cap may comprise one or more engagement tabs. The cap may be provided with two engagement tabs. The one or more engagement tabs may extend axially from the open end. The engagement tabs may be configured to flex radially.

The cap and movable insert may comprise corresponding engagement means. The engagement tabs may engage with the inserter tip. The inserter tip may comprise projections. The moveable insert may comprise recesses. The corresponding engagement means, e.g. projections and recesses, may be configured to remain engaged when subjected to the first predetermined force and configured to disengage when subject to the second predetermined force.

The engagement means may comprise projections on the movable insert and corresponding apertures on the cap. When the cap and movable insert are engaged the projections may be arranged in the apertures. The cap may be deformed to remove the projections from the apertures. The cap may be removed to decouple from the movable insert.

The catheter assembly may comprise a hermetic seal for preserving the sterility of the internal volume prior to use. In the first position the cap may provide a sterile seal for a cavity within the catheter assembly body.

When in a sealed configuration, the hermetic seal may be intact. When in a primed or wetting configuration, the hermetic seal may be broken such that the cavity within the catheter assembly body is at least partially open to the external air such that sterile environment may be considered breached.

The catheter tube may be functionalised. For example it may comprise, be integrated with, or be coated with a hydrophilic component (e.g. a hydrophilic polymer). The hydrophilic component serves to reduce friction further upon application of the wetting agent. At least an external surface of the catheter tube may be functionalised, e.g. the hydrophilic component may be provided on at least an external surface of the catheter tube (which is in contact with the urethra in use). The catheter may comprise a main flow path for the passage of urine. The main flow path may extend along and define a longitudinal axis of the catheter. The main flow path may be provided by a wall of catheter tube. The main flow path may have a proximal inlet at an insertion end of the catheter, and a distal outlet.

The catheter may comprise an outlet body. The outlet body may incorporate the terminal end of the catheter tube. The outlet body may comprise the external handling surface of the catheter. The outlet body may comprise one or more flow enhancing features for aiding the flow from catheter tube. The one or more flow enhancing features may comprise a funnel, for example.

The outlet body may comprise or be referred to as a connector which connects the outlet end, e.g. a funnel and/or the external handling features, and the catheter tube.

The sheath may be a retractable sheath. The retractable sheath may be configured to be retracted during insertion of the catheter such that it provides a temporary enclosure around the catheter tube prior to insertion.

According to a further aspect of the present disclosure there is provided a method of assembling a catheter wetting agent storage chamber, the catheter wetting agent storage chamber defined by a first end wall, a second end wall and a radially outer wall extending along a longitudinal axis between the two, wherein the catheter wetting agent storage chamber comprises: a first section comprising a first part of the outer wall and the first end wall, a second section comprising the second end wall and a second part of the outer wall; wherein one of the first and second sections comprises at least one projection and the other of the first and second sections comprises a corresponding recess in an outer surface of the outer wall; the method comprising axially inserting one of the first and second section into the other such that the projection axially projects into the recess.

Optional features set out above may apply to any aspect of the invention.

### DETAILED DESCRIPTION OF THE INVENTION

In order that the invention may be more clearly understood one or more embodiments thereof will now be described, by way of example only, with reference to the accompanying drawings, of which:

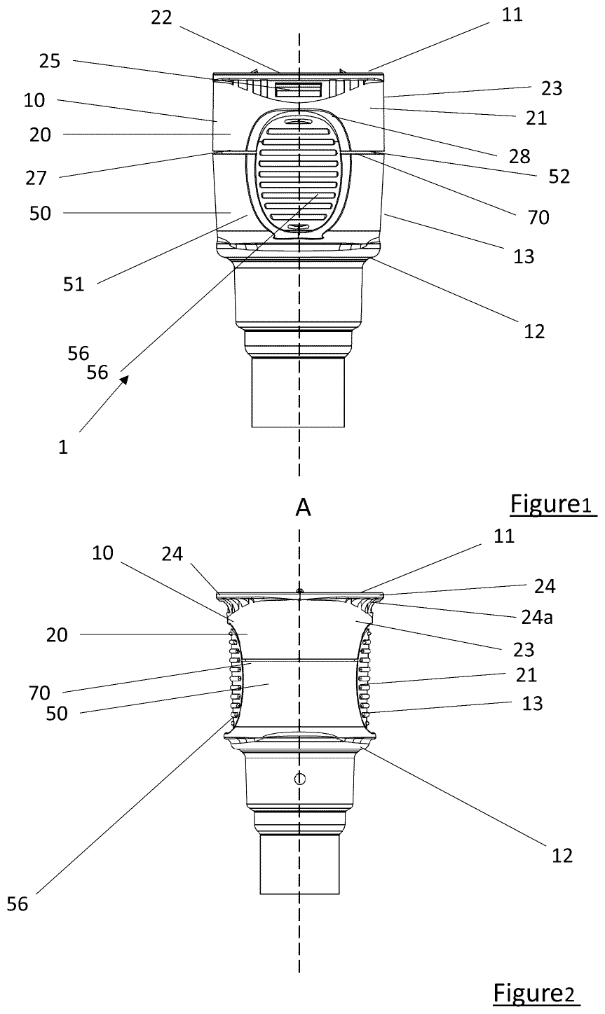

FIG. 1 shows an orthographic side view of a catheter wetting agent storage chamber according to an embodiment of the disclosure;

FIG. 2 shows an orthographic front view of the catheter wetting agent storage chamber of FIG. 1

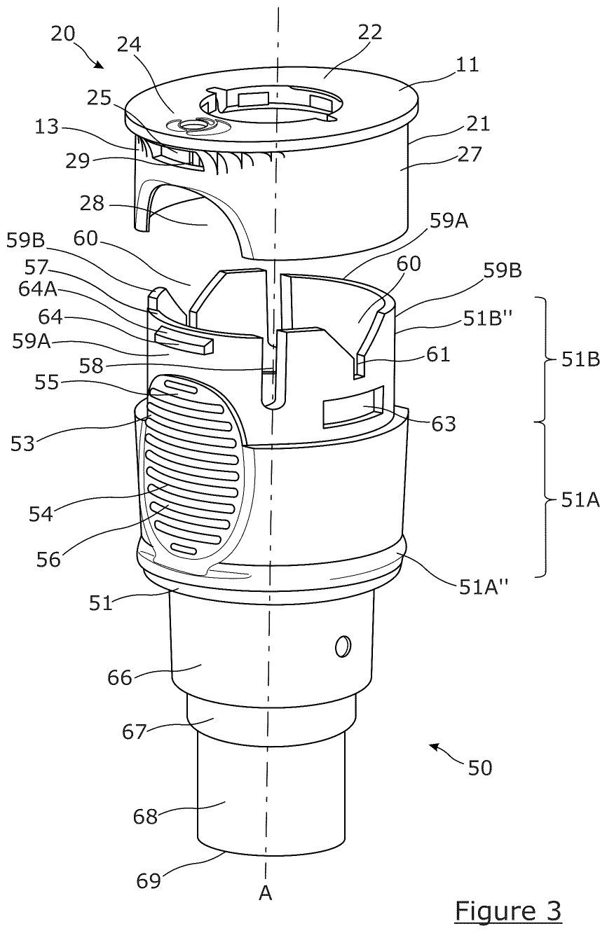

FIG. 3 shows an exploded perspective view of the catheter wetting agent storage chamber of FIG. 1;

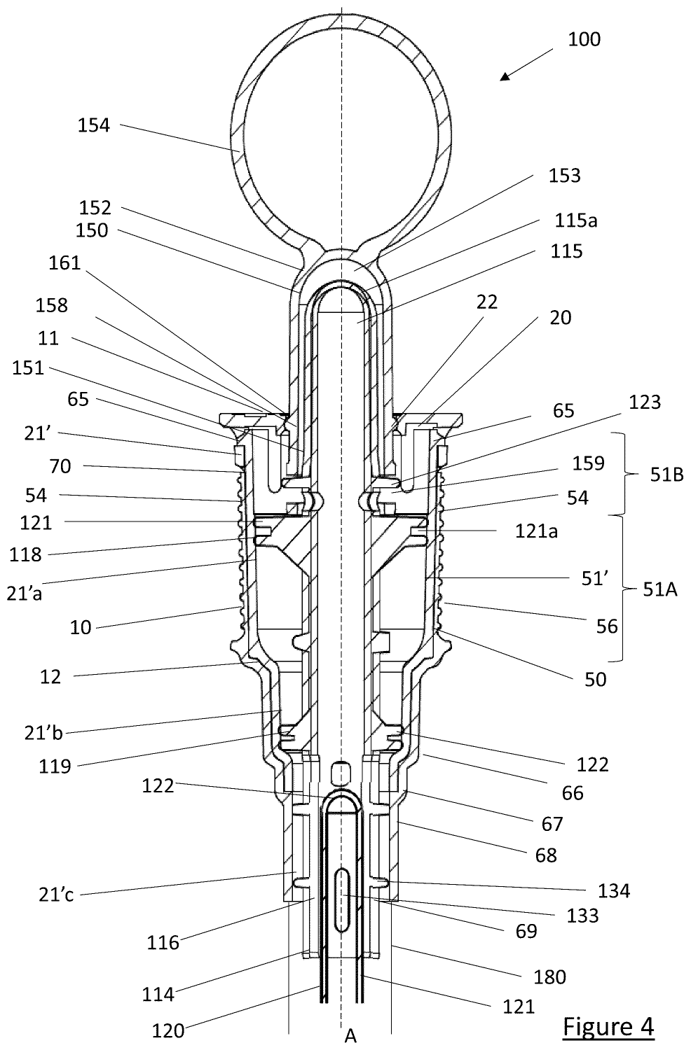

FIG. 4 shows a longitudinal cross-section of a catheter assembly comprising the catheter wetting agent storage chamber of FIG. 1 in a closed configuration; and

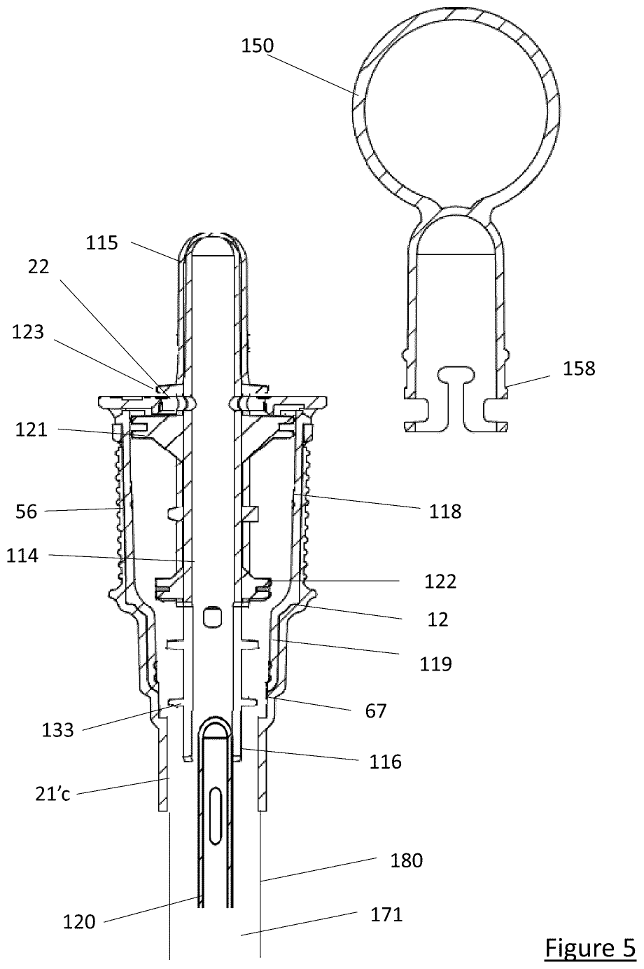

FIG. 5 shows a longitudinal cross-section of the catheter assembly of FIG. 4 in a deployed configuration.

With reference to FIGS. 1-5, an exemplary catheter wetting chamber 1 according to the present invention is described.

Referring to FIGS. 1 to 3, the catheter wetting chamber 1 comprises an external housing 10 with an overall cylindrical shape with a first end wall 11, a second end wall 12, and a radially outer wall 13. The external housing 10 is formed in two parts, a first, upper, section 20 and a second, lower, section 50. It will be appreciated that the terms “upper” and “lower” are provided only to aid the description and do not infer any orientation. To further aid the description of the wetting chamber, there is shown a longitudinal axis A. Unless stated otherwise all positions and axes are relative to these.

The upper section 20 comprises a first portion 21 of the radially outer wall 13 terminating at one end with the first end wall 11 and open at second end. Arranged at the centre of the first end wall 11 is a circular insertion end aperture 22 through which a catheter (not shown) can pass. The first end wall 11 may have an elliptical shape, the semi-minor axis of which has the same length as the radius of an outer surface 23 of the first portion 21 of the radially outer wall 13. The length of the semi-major axis is greater than the radius of the outer surface 23 of the first portion 21 of the radially outer wall. As the first portion 21 radially outer wall 13 is circular, where the first end wall 11 is elliptical, the first end wall 11 projects radially out beyond the outer wall 13 at two points to form tabs 24 to aid gripping the wetting chamber as will be explained in greater detail below. The tabs 24 may be reinforced by a plurality of ribs 24a which extend from the ribs 24 to the radially outer wall 13.

Arranged below both tabs 24 are rectangular slots 25 (only one is shown but the skilled person will appreciate that a further slot is provided on the opposing side) extending through the first portion 21 the radially outer wall 13. The slots 25 have a long axis extending circumferentially around the radially outer wall and a short axis extending axially along the radially outer wall 13. The rectangular slots 25 form a first part of an engaging means 65 between the upper 20 and lower 50 sections as will be described in detail below. In other embodiments the rectangular slots 25 may not extend through the radially outer wall, but may instead be a recess set in one side of the wall.

As noted above, the second end of the first portion 21 is open, the radially outer wall 13 terminating with an edge 27. On opposing sides of the edge 27 aligned with the tabs 24 are provided two recesses 28 (only one is shown but the skilled person will appreciate that a further recess is provided on the opposing side). The recesses 28 may have a “D” shaped profile and extend through the first portion 21 of the radially outer wall 13.

Arranged on an inner surface of the radially outer wall 13 are two axially extending ribs 29, the two ribs 29 are positioned on opposing sides of the radially outer wall 13 midway between the two recesses 28. The two axially extending ribs 29 extend from the first end wall 11 halfway to the open second end, the two ribs form a first part of an alignment mechanism described in detail below.

The lower section 50 comprises a second portion 51 of the radially outer wall 13, terminating at one end with the second end wall 12 and open at the opposing, open, end 52. The second portion 51 of the radially outer wall 13 is provided in two parts, an external wall part 51A and an overlap part 51B. The external wall part 51A is proximal to the second end wall 12 and the overlapping part 51B is distal to the second end wall 12. The diameter of inner faces of both parts is the same so as to provide a single continuous face of the inner wall 51′. The overlapping part 51B has a thinner wall than the external wall part 51A, this results in radially outer faces of the external wall part 51A″ and overlapping part 51B″ being separated by a step 53, with the outer face of the overlapping part 51B″ being radially inward from the outer face of the external wall part 51A″.

Extending axially over the step 53 from the external wall part 51A to the overlapping part 51B are two projections 54. The projections have the same radially outer diameter as the external wall part 51A and thus form a continuation of this part into the overlapping part 51B. The projections have a curved distal end 55, that is the middle of the projections extend into the overlapping part 51B to the greatest extent. The curved distal end 55 has a corresponding profile to the recesses 28 in the first portion 21, the two features being shaped to engage together as described in detail below.

Gripping features 56 are provided on radially outer faces of the projections 54, the gripping features 56 extend beyond the projections 54 onto the outer face of the external wall part 51A″. The gripping features 56 define an area with an oval shape a portion of the edge of which in contiguous to the curved distal end 55. In some embodiments the gripping features 56 may be integrally formed with the outer face of the external wall part 51A″, in other embodiments the gripping features 56 may be formed of a different material, for example one which provides increased grip when wet.

The radially outer face of the overlapping part 51B″ has a diameter equal to a radially inner wall 21′ of the first portion 21 such that the overlapping part 51B can be disposed within the first portion 21 as will be described in detail below.

Extending axially from an edge 22 of the open end 52 of the second portion 51 are four axial slots 58. The axial slots 58 are equidistantly spaced about the edge 57 and the two slots 58 adjacent to each projection 54 are equally separated. The four axial slots 58 separate the open end 53 of the second portion 51 into four tabs 59, the projections 54 extend into two opposing attachment tabs 59A. Between the two attachment tabs 59A are two alignment tabs 59B, the alignment tabs 59B each comprise a triangular notch 60 tapering away from the open end 53 and ending in a point, further extending from the point is an axially extending slit 61. The axially extending slit 61 forms a second part of the alignment mechanism, the operation of which is detailed below.

Between both of the axially extending slits 61 and the external wall part 51A are provided a circumferentially extending rectangular slot 63. The slots 63 provide fluid communication between the interior and exterior of the catheter wetting agent storage chamber 1 as will be described in detail below.

At a distal end of both of the attachment tabs 59A, proximate to the edge 57 of the open end 52 of the second portion, there is provided a protuberance 64 with a chamfered edge 64A facing the edge 57. The two protuberances 64 are configured to engage with the rectangular slots 25 and form the second part of the engagement means 65.

On an opposing side of the second end wall 12 to the second portion 51 of the radially outer wall 13 are three concentrically arranged tubular sections, a first tubular section 66, and a second tubular section 67 and a third tubular section 68. The first tubular section 66 is proximal to the second end wall 12 and has a diameter less than that of the radially outer wall 13. The second tubular section 67 is arranged beyond the first tubular section 66 and has a diameter less than the first tubular section 66. The third tubular section 68 is arranged beyond the second tubular section 67 and has a diameter less than the second tubular section 67. A chamfered section is provided between each tubular section to reduce the diameter. The distal end of the tubular sections is open, providing a lower opening 69 which is aligned with the insertion end aperture 22.

With reference to FIGS. 3 and 4 a method of assembling the wetting agent storage chamber 1 is described. The upper section 20 and lower section 50 are rotated such that the recesses 28 on the upper section 20 align with the projections 54 on the lower section 50. The overlapping part 51B of the lower section 50 is inserted axially into the upper section 20. Any misalignment of the two sections may be corrected for by the alignment mechanism comprising the notches 60 in the alignment tabs 59B which will engage with the corresponding ribs 29 on the inner face of the first portion 21 of the radially outer wall 13 to rotate the two sections 20,50 relative to one another until they are in the correct alignment.

As the two sections 20,50 are brought together the projections 54 slot into the recesses 28 and the protuberances 64 contact with the edge 27 of the open end of the first portion 21 of the radially outer wall 13 and cause a radially inward deflection of the two attachment tabs 59A. As the two sections 20,50 are almost completely connected the wetting agent storage chamber is filled with a wetting agent (which in this embodiment is water). The wetting agent is supplied through the rectangular slots 63. As the two section 20,50 continue to be brought together the protuberances 64 will enter their respective rectangular slots 25 and the two attachment tabs 59A will return to their original positions. The protuberances 64 will engage with their respective slots 25 and thereby hold the two sections 20,50 together to form the wetting agent storage chamber 1 and the first portion 21 of the radially outer wall will seal the slots 63.

The provision of corresponding projections 54 and recesses 28 provide a discontinuous join line 70 between the two sections 20,50 of the storage chamber. That is the join line 70 between the two sections visible to a user on the radially outer surface 23 is not a circumferentially extending circular line, but instead has an also has an axial component. This provides a visual indicator to the user that the deployment of the catheter, in particular the catheter wetting agent storage chamber 1 does not require rotation.

The catheter wetting agent storage chamber 1 may be provided as part of a catheter assembly 100 such as that shown in FIG. 4. The catheter assembly 100 comprises the catheter wetting agent storage chamber 1, a movable insert 114, a catheter 120, a cap 150 and a sheath 180.

The catheter assembly 100 may be configured such that the catheter 102 may be wetted prior to being withdrawn from the external housing 10. A wetting mechanism may be provided within the external housing 10.

Within the external housing 10 and projecting from each open end 22,69 is the movable insert in the form of an inserter 114, the inserter 114 having an overall tubular shape and comprising an inserter tip 115 and a catheter gripper 116. The external housing 10 and inserter 114 are concentrically arranged such that the inserter 114 is located within the external housing 10 in a radially nested configuration. The inserter tip 115 is provided at a first end of the inserter 114 and closes the tubular body with a hemispherical end 115a, the hemispherical end 115a is provided with one or more apertures or openings such as slits (not shown) which allow the end 115a to be opened to allow passage of a catheter therethrough. The opposite end of the inserter 114 is provided with the catheter gripper 116, the structure and operation of which will be elaborated upon below.

In this embodiment the inserter 114 is arranged within the external housing 10 such that in the storage position (as will be elaborated upon below) the inserter tip 115 extends out of the first end wall 11 and the catheter gripper 116 extends out of the second end wall 12 of the external housing 10.

In this embodiment the cap 150 has a tubular shape with an open end 151 and a closed end 152, the interior of the cap 150 defines a cavity 153 shaped to receive the inserter tip 115. The cavity may be sized and shaped to closely conform to the inserter tip 115. On the exterior of the cap 150 at the closed end 152 there is provided a grab loop 154 by which a user can grip the cap 150. The cap 150 extends through the inserter end aperture 22 where a housing seal 161 is provided between the aperture 22 and the cap 150 to hermetically seal the wetting agent storage chamber.

The catheter 120 is of a type known to the art, comprising a catheter tube 121 with an insertion end 122 and an opposing drainage end (not shown). The catheter tube 121 has a longitudinal axis which corresponds to the longitudinal axis A of the catheter wetting agent storage chamber. Proximate to the insertion end 122 there may be provided one or more drainage apertures 133, in this embodiment the drainage apertures 133 are oval in shape with the major axis being parallel with the longitudinal axis A. It will be appreciated that the size and shape of the drainage apertures 133 may differ.

The exterior surface of the catheter tube 121 may be, as is known in the art, functionalised such that when wetted by the wetting agent the co-efficient of friction of the catheter tube 121 is reduced. The outer surface of the catheter tube 121 may be comprised of, or coated in, a functionalising material; for example the outer surface of the catheter tube 121 may have hydrophilic properties. The hydrophilic properties serve to reduce the coefficient of friction of the outer surface when the wetting agent is introduced.

The catheter 120 and inserter 114 are concentrically arranged such that the insertion end 122 of the catheter 120 engages with the catheter gripper 116 with the distal end of the catheter 120 extending away from the inserter 114 as will be described in further detail below.

Arranged at the catheter end 112 of the external housing 10 is the sheath 180. The sheath 180 extends away from the external housing 10 so as to surround at least a part of the catheter tube 120. The sheath 180 is formed of a flexible material. In some embodiments, the sheath 180 is attached to the external housing in such a way as to provide a leak resistant seal. In some embodiments the distal end of the sheath (not shown) may be joined to a pouch for collection of the fluid discharged from the catheter 120, alternatively the sheath 180 and pouch may be integrally formed. The sheath may allow for fluid communication from the external housing 10 to the pouch.

The radially inner wall 21′ of the external housing 10 is provided with a step proximate to the catheter end 112 such that the diameter of the inner wall 21′ is reduced proximate to the catheter end 12, and radially inward of the tubular sections 66,67,68. The first, wider, portion 21′a of the inner wall 21′ provides a first sealing surface 118 and the second, narrower, portion 21′b of the inner wall 21′ provides a second sealing surface 119. The internal diameter of the inner wall 21′ is further reduced at the catheter end 112 edge by the third tubular section 68 to form a third, narrowest, portion 21′c.

The inserter 114 is radially nestled with the external housing 10. In this embodiment to ensure the alignment of the inserter 114 in the external housing the inserter 114 is provided with two pairs of ribs 121,122. In other embodiments the external housing 10 may be provided with projections such as ribs.

In this embodiment a first (upper) pair of ribs 121 project circumferentially around, and radially outward, from the inserter 114 at a point approximately one third of the distance from the inserter tip 115a to the catheter gripper 116. The first pair of ribs 121 are sized such that they extend radially to the first sealing surface 118 of the external housing 10, the first pair of ribs 121 are formed from a single broader projection, the outer circumference of which is provided with a groove 121a with a depth equal to have the radial extent of the projection, this separates the projection into two equally sized ribs 121.

In this embodiment a second (lower) pair of ribs 122 project circumferentially around, and radially outward, from the inserter 114 at a point proximate to the catheter gripper 116. The second pair of ribs 122 are sized such that they extend radially to the second sealing surface 119 of the external housing 10. The two ribs 122 of the second pair project separately from the inserter 114.

In this embodiment proximate to the first pair of ribs 121 are provided two cap tabs 123, the cap tabs 123 are two projections on opposing sides of the inserter 114. The cap tabs 123 have a rectangular base with the major edge normal to the longitudinal axis A of the catheter assembly 10.

In this embodiment the open end 151 of the cap 150 is provided with two engagement tabs 158. The two engagement tabs 158 are each provided with a rectangular aperture 159 the long axis of which extends circumferentially around the cap. The apertures 159 are sized and shaped to receive the cap tabs 123 of the inserter 114. About the apertures 159 the thickness of the wall of the connector part 141 increases with a chamfered step provided on the radially outer surface.

When provided in the sealed configuration, for example for storage and shipping the inserter 114 is provided in a retracted position within the external housing 10. The first and second pairs of ribs 121, 122 bear against the sealing surfaces 118,119 of the radially inner wall 21′ to align the inserter 114 within the external housing 10 such that they are radially nested.

Together the inner wall 21′ of the external housing 10, outer surface of the inserter 114, and ribs 121,122 retain a wetting agent (which in this embodiment is water) within the wetting agent storage chamber 1, the inserter 114 and external housing 10 defining the radial extent and the upper and lower ribs 121,122 the axial extent.

In the sealed configuration the inserter 114 is inserted to the maximum extent into the external housing 10, and the second pair of ribs 122 abut the chamfered section 67 restricting any further insertion.

The cap 150 is arranged on the inserter 114 such that the inserter tip 115 is within the cavity 153. The cap tabs 123 project into the apertures 159 in the engagement tabs 158 and the open lip 156 abuts the first pair of ribs 121. The catheter gripper 116 is provided with a small bulge 134 which bears against the narrowest portion 21′b of the inner wall. This bearing on the small bulge 134 deforms the gripper end 116 compressing it such that it engages with the insertion end 122 of the catheter 120 to retain the catheter 120 in place.

With reference to FIGS. 4 and 5 a method of deploying and using the catheter is described. When the catheter 120 is to be deployed for use, the user can grip the external housing 10 by the gripping features 56 with one hand and by the cap 150, for example by the grab loop 154 where present, with the other hand. Prompted by the discontinuous join line 70 the user knows the catheter 120 is not deployed by a rotational motion and instead pulls the cap 150 axially relative to the external body 10.

During deployment, the catheter assembly 100 is arranged generally vertically, with the cap 150 uppermost (as shown in FIG. 4), so that the catheter 120 is below the wetting agent storage chamber 1. Gripping the gripping features portion 56 and the grab loop 154 a user pulls the cap 150 axially away from the external housing 10. As the cap 50 is displaced axially away from the external housing 10, the aperture lip 42a disengages from the indent 63 thereby breaking the housing seal 161; the catheter assembly 10 is no longer hermetically sealed.

With continued axial displacement, the cap tabs 123 abut the edge of their corresponding apertures 159, with this the inserter 114 begins to be axially displaced. As the inserter 14 is displaced the small bulge 133 passes the chamfered section 67 and becomes axially misaligned with the third inner wall portion 21′c. As the small bulge 133 is no longer pressed against the third inner wall portion 21′c the gripper end 116 Is no longer compressed and relaxes in a radially outward manner providing a separation between the gripper end 116 and the catheter 120. This increase in separation results in the gripper end 116 disengaging with the catheter 120 such that the catheter is free to be moved and the entire functionalised surface of the catheter tube 121 is exposed. Movement of the inserter 14 within the external housing 10 is guided by the first and second rib pairs 121,122 bearing against and moving along the sealing surfaces 118,119.

In this embodiment as the cap 150 and inserter 114 continue to be displaced, the second pair of ribs 122 pass the second wall 12 and become axially misaligned with the second scaling surface 119. A flow path is thereby opened between the wetting agent storage chamber 10 and a cavity defined between the catheter 120 and sheath 180, in this regard the interior of the sheath can be considered a wetting chamber. The wetting agent flows from the wetting agent storage chamber 10 to the catheter 120 around the second rib pair 122. The wetting agent is replaced in the wetting agent storage chamber 10 with air via the rectangular slot 63. In this embodiment any excess wetting agent which does not contact the catheter flows through the sheath 80 and is discharged into the pouch.

In this embodiment as the storage chamber 10 is being opened the engagement tabs 158 and thus cap tabs 123 are drawn through the insertion end aperture 22. Although the cap tabs 123 are wider than the aperture 22 the chamfered step deforms the aperture 22 increasing the diameter, this allows the cap tabs 123 to pass through the aperture 22. The inserter 114 continues to move axially until the first pair of ribs 121 abut the first end wall 11 at which point the engagement tabs 158 have passed through the aperture 22 and are outside the external housing 10. Throughout the entire movement of the inserter 114 the first pair of ribs 121 remain in contact with the first sealing surface 118 ensuring no wetting agent leaks from the first end 11 of the external housing 10.

As the engagement tabs 158 are no longer radially nested within the aperture 22 or external housing 10 one or both can be deformed (either plastically or resiliently, depending upon the material) such that the cap tabs 123 are no longer within the cap apertures 159 and the cap can be removed as seen in FIG. 5.

With the cap 150 removed, the inserter tip 115 is exposed, gripping features 56 the user can introduce the inserter tip 115 into the urethra, the extent to which this can be done being limited by the cap tabs 123. Though the inserter 114 is movable it does not retract back into the external housing 10 when introduced into the urethra because the cap tabs 123 cannot return through the insertion end aperture 22.

Once the inserter tip 15 is in place the user can grip the catheter 120 through the sheath 180 and introduce it into the urethra and drain the bladder in the usual manner.

The one or more embodiments are described above by way of example only. Many variations are possible without departing from the scope of protection afforded by the appended claims.

For example, whilst the embodiments are all female intermittent urinary catheters, with an exemplary length of between 90 mm to 200 mm. e.g. between 130 mm and 155 mm, such as about 135 mm and the catheter assemblies have a length corresponding to the length of the catheter, such as a closed length of the casing of between 2 mm and 10 mm longer than the length of catheter (e.g. 10-25 cm; between 140 mm and 165 mm, such as 142 mm), it is considered that teachings could be applied to male urinary intermittent catheters (which are typically longer) or even other types of catheter. Similarly, although the embodiments have functionalised hydrophilic surfaces which become slippery when wetted with a wetting agent such as water, the wetting agent could be a lubricant instead.

1. A catheter wetting agent storage chamber defined by a first end wall, a second end wall and a radially outer wall extending along a longitudinal axis between the two, wherein the catheter wetting agent storage chamber comprises:
a first section comprising a first part of the outer wall and the first end wall;
a second section comprising a second part of the outer wall and the second end wall;
wherein one of the first and second section is disposed radially inside the other section such that the sections overlap axially;
and wherein one of the first and second sections comprises at least one projection which extends axially into a corresponding recess in an outer surface of the outer wall of the other of the first and second sections.

2. A catheter wetting agent storage chamber according to claim 1 wherein the second section is disposed inside the first section and the second section comprises the at least one projection.

3. A catheter wetting agent storage chamber according to claim 1 or 2 wherein the catheter wetting agent storage chamber comprises an equal number of projections and recesses.

4. A catheter wetting agent storage chamber according to any preceding claim wherein the catheter wetting agent storage chamber comprises two projections.

5. A catheter wetting agent storage chamber according to any preceding claim wherein the or each recess extends radially through the outer wall of the corresponding section.

6. A catheter wetting agent storage chamber according to any preceding claim wherein the or each projection has the same profile as the or each recess.

7. A catheter wetting agent storage chamber according to any preceding claim wherein the projection is provided on an outer surface of the outer wall.

8. A catheter wetting agent storage chamber according to any of claims 2 to 7 wherein the second part of the outer wall has a distal section with a first diameter and a proximal section with a second diameter, wherein the first diameter is less than the second diameter.

9. A catheter wetting agent storage chamber according to claim 8 wherein the first diameter is equal to an inner diameter of the first wall.

10. A catheter wetting agent storage chamber according to claim 8 or 9 wherein the second diameter is equal to an outer diameter of the first wall.

11. A catheter wetting agent storage chamber according to any of claims 8 to 10 wherein the projections have the second diameter.

12. A catheter wetting agent storage chamber according to any preceding claim wherein the or each projection has a curved distal end.

13. A catheter wetting agent storage chamber according to any preceding claim wherein the or each projection has a textured outer surface.

14. A catheter wetting agent storage chamber according to any preceding claim wherein the first and second sections engage with a snap fit.

15. A catheter wetting agent storage chamber according to claim 14 wherein the snap fit comprises a radially extending projections in one of the first and second sections and the other of the first and second sections comprises a corresponding recess.

16. A catheter assembly comprising a catheter and a catheter wetting agent storage chamber defined by a first end wall, a second end wall and a radially outer wall extending along an longitudinal axis between the two, wherein the catheter wetting agent storage chamber comprises:
a first section comprising a first part of the outer wall and the first end wall;
a second section comprising a second part of the outer wall and the second end wall;
wherein one of the first and second section is disposed radially inside the other section such that the sections overlap axially;
and wherein one of the first and second sections comprises at least one projection which extends axially into a corresponding recess in an outer surface of the outer wall of the other of the first and second sections.

17. A catheter assembly according to claim 16 wherein the catheter wetting agent storage chamber is the catheter wetting agent storage chamber according to any of claims 1 to 15.

18. A catheter assembly according to claim 16 or 17 further comprising a movable insert axially movable between a first position and a second position.

19. A catheter assembly according to claim 18 wherein in the first position the catheter wetting agent storage chamber is sealed, and in the second position the catheter wetting agent storage chamber is open.

20. A catheter assembly according to claim 18 or 19, wherein the movable insert comprises a rib, wherein in the first position the rib aligns with a corresponding sealing surface on an inner wall of the catheter wetting agent storage chamber, and in the second position the rib is axially misaligned with the corresponding sealing surface.

21. A catheter assembly according to any of claims 18 to 20 further comprising a wetting agent, wherein in the first position the catheter is not in contact with the wetting agent and in the second position the wetting agent is in contact with the catheter.

22. A method of assembling a catheter wetting agent storage chamber, catheter wetting agent storage chamber defined by a first end wall, a second end wall and a radially outer wall extending along a longitudinal axis between the two, wherein the catheter wetting agent storage chamber comprises:
a first section comprising a first part of the outer wall and the first end wall;
a second section comprising a second part of the outer wall and the second end wall;
wherein one of the first and second sections comprises at least one projection and the other of the first and second sections comprises a corresponding recess in an outer surface of the outer wall, the method comprising:
axially inserting one of the first and second section into the other such that the projection axially projects into the recess.

23. A method of assembling a catheter wetting agent storage chamber according to claim 22, wherein the catheter wetting agent storage chamber is a catheter wetting agent storage chamber according to any of claims 1 to 15.
